(19) United States
(12) Patent Application Publication
(10) Pub. No.: US 2004/0002079 A1
(43) Pub. Date: Jan. 1, 2004

(54) Sodium-phosphate cotransporter in lithium therapy for the treatment of mental illness

(76) Inventors: Robert B. Gunn, Atlanta, GA (US); Richard T. Timmer, Atlanta, GA (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/314,790

(22) Filed: Dec. 9, 2002

Related U.S. Application Data
(63) Continuation of application No. 09/380,164, filed on Aug. 26, 1999, now abandoned.

(30) Foreign Application Priority Data
Feb. 11, 1998 (WO) PCT/US98/02875

(51) Int. Cl. C12Q 1/68; C07H 21/04; C12P 21/02; C12N 5/06; C07K 14/705

(52) U.S. Cl. 435/6; 435/69.1; 435/235.1; 435/320.1; 435/325; 530/350; 536/23.5

(57) ABSTRACT

The sodium-phosphate cotransporter existing on virtually every human cell is identified as the same protein as the lithium-sodium counterttransporter, and is suitable for diagnostic assays for mental illnesses susceptible to lithium therapy, including manic depression.

### BACKGROUND OF THE INVENTION

The subject matter of this present invention was developed in part by one or more grants of the United States Government, NIH HL-28674 and NIH HL-08989.

Na—PO₄ cotransport is the primary mechanism for the regulation of total body phosphate balance. It mediates both the gastrointestinal uptake and renal reabsorption of PO₄. Whereas 70% (high phosphate diet) to 85% (low phosphate diet) of dietary phosphate is absorbed by the GI tract the major control is in proximal reabsorption of the kidney of about 70% of the filtered load and in distal discretionary reabsorption of some fraction of the remainder. Na—PO₄ cotransport has been found in the plasma membrane of every mammalian cell examined. In cell membranes it is the principal mechanism for PO₄ uptake against the usually negative membrane potential and makes the cytoplasmic and extracellular concentrations approximately equal (±3-fold). Intracellularly, phosphate metabolism includes most important biological molecules: nucleotides, DNA, RNA, glycolytic intermediates, phospholipids, and most proteins through regulatory or structural phosphorylations. Within the mitochondrial membrane Na—PO₄ cotransport is essential for ATP synthesis.

Na—PO₄ cotransporters exist in the kidney, as two isoforms, type I and type II. A related cotransporter is also present in liver where the protein has been partially purified, reconstituted in liposomes, and expressed in oocytes from liver mRNA. Its function in rat hepatocytes in primary culture is stimulated by insulin. Na—PO₄ cotransport activity has been extensively characterized in the duodenum/upper jejunum of many higher vertebrates. Neither the liver nor intestinal forms of the cotransporter have been cloned from mammalian sources. In contrast to mammals, teleosts appear to have an isoform of the Type II transporter present in the intestine. Alterations in intestinal Pᵢ reabsorption appear to be related to 1,25-dihydroxy-vitamin D₃ status and/or dietary Pᵢ intake. Recently a brain-specific cDNA, designated BNPI, has been cloned that appears to encode a Na—PO₄ cotransporter and is 32% identical to the rabbit renal cotransporter, NaPi-1. The brain transporter is specific to the brain and mRNA transcripts are found in the neurons of the cerebral cortex, hippocampus, and cerebellum. The neuronal transporter is also found in peripheral nerves and transports arsenate and Li⁺ can substitute for Na⁺. Evidence suggests at least three, possibly four, distinct isoforms of the cotransporter, the renal types I and II, as well as the brain-specific form which may represent a special type. The erythrocyte form represents a third type of Na—PO₄ cotransporter, which applicants have discovered is also a retroviral receptor.

Retroviruses require specific cell-surface receptors for cell recognition and infection. Two widely expressed mammalian retrovirus receptors PiT-1 (Glvr-1; Genbank L20859, U.S. Pat. No. 5,414,076) and PiT-2 (Ram-1; Genbank L19931, U.S. Pat. No. 5,550,221) have been cloned and shown to share 30% homology with Pho-4⁺, a phosphate uptake gene in Neurospora crassa and when these two mammalian genes are expressed in oocytes they induce sodium dependent phosphate cotransporter activity. Also, a murine cationic amino acid transporter has been shown to be a retrovirus receptor, thus, indicating there are at least two classes of transporters that are retrovirus receptors. The two sodium phosphate cotransporters/retrovirus receptors (PiT-1 and PiT-2) are widely expressed in tissues and cells (thymus, marrow, lung, liver, heart, kidney, muscle and brain) and appear to be the ubiquitous housekeeping sodium-phosphate cotransporters that every cell requires in order to maintain the intracellular concentration of phosphate above electrochemical equilibrium. Furthermore the cotransporter/receptor isoforms in different species (human and mouse; rat and hamster) together with the differences in retroviral envelope proteins define the species specificity for susceptibility to infection by each retrovirus. The ability to transfect cell lines from one species with the transporter/receptor isoform from another species and/or alter the envelope protein provides novel model systems and the means to design vectors that allow increased gene transfer into human hematopoietic progenitor cells and other cells.

Two sodium-phosphate cotransporters, PIT-1 and PiT-2, are found in most cells. A third cotransporter BNPI There are different isoforms of these three genes in different people. cDNA and cRNA probes to PiT-1 or to PiT-2 and their mRNA products and antibodies to these proteins distinguish between individuals who are responders or non-responders to lithium treatment.

Applicants have discovered that the sodium-phosphate cotransporter is the same cell membrane protein as the lithium-sodium countertransporter. This discovery has important implications for the diagnosis and therapy of patients in need of lithium for the treatment of manic depression. The present invention provides a readily performed diagnostic test to evaluate patient status, by measuring a combination of sodium, phophate or lithium flux in an in vitro membrane-based translation system.

Applicants have identified the gene product of PiT-1 as the lithium-sodium countertransporter across cell membranes. The PiT-1 gene product is the erythrocyte isoform. Probes for this gene distinguish between responders and non-responders to lithium treatment.

Applicants have also identified the lithium-sodium countertransporter as the physiological mechanism for the extrusion of lithium from cells. It regulates the cell concentration of lithium. The activity of this transporter determines the therapeutic effect of lithium. This invention provides a simple molecular biological test for the ability of cells to extrude lithium. Presently, the only test to determine the activity of a lithium transporter is a laboratory measurement of lithium flux into or out of cells using chemical assays for lithium. See, e.g., Sarkadi, B. et al., J. Gen. Physiol. 72: 249 (1978).

The lithium-sodium countertransporter has significance for determining the responsiveness of humans with mental disorders to treatment with lithium salts. At present about half of patients treated with lithium do not improve. There are no techniques at present to diagnose whether a patient will be helped by lithium treatment, except by a time-consuming therapeutic trial. The diagnostic test of the present invention allows genetic screening to predict whether a patient will respond to lithium transport. The test is also a screen for susceptibility to and extent of manic depressive illness. Further, the test is suitable to screen newborns in families with depression for their potential to develop the illness and whether they can respond to lithium treatment.

### BRIEF DESCRIPTION OF THE INVENTION

The sodium-phosphate cotransporter is identified as the same protein as the lithium-sodium countertransporter, and is suitable for diagnostic assays for mental illnesses susceptible to lithium therapy, including manic depression. Various methods for evaluating the flux of lithium and other cations in appropriate cells are also disclosed, including reticulocytes.

### DESCRIPTION OF THE FIGURES

FIG. 1 is a schematic diagram of a cell 1 showing that the sodium-phosphate cotransporter is the same cell membrane protein as the sodium-lithium countertransporter.

### DEFINITIONS AND ABBREVIATIONS

Pᵢ Concentration of inorganic phosphate

### DETAILED DESCRIPTION OF THE INVENTION

The present invention relates to a purified DNA molecule coding for a lithium-sodium countertransporter. It also relates to a purified DNA molecule coding for an amino acid sequence selected from the group consisting of hPiT-1, hPiT-2, and hBNPI, said molecule useful for measuring lithium-sodium countertransport in human cells. Specifically, the present invention relates to a novel utility for the sequences identified as SEQ.ID.NO.: 1, SEQ.ID.NO.: 2, SEQ.ID.NO.: 3, SEQ.ID.NO.: 4, SEQ.ID.NO.: 5, and SEQ.ID.NO.: 6.

In one embodiment of the present invention, applicants show that the human amphotrophic retrovirus receptor is useful as a lithium-sodium countertransporter, including the sequences identified as SEQ.ID.NO: 1, SEQ.ID.NO.:2, SEQ.ID.NO.: 3, SEQ.ID.NO.: 4, SEQ.ID.NO.: 5, and SEQ.ID.NO.: 6.

In another embodiment of the present invention there is provided a first method of evaluating sensitivity to lithium therapy in manic depressive patients, comprising the steps of

(a) providing a sample of patient blood;

(b) extracting from the blood sample the patient's DNA;

(c) subjecting the DNA to hybridization with primers specific for any sequence coding for lithium-sodium countertransporter;

(d) polymerizing said sequences, to give polymerized sequences;

(e) amplifying said polymerized sequences, to give an amplified sample of patient sequences;

(f) digesting the amplified sample with one or more restriction endonucleases suitable for mapping sites on the DNA indicating susceptibility to lithium therapy.

In another embodiment of the present invention, there is provided a second method of evaluating sensitivity to lithium therapy in manic depressive patients, comprising the steps of

(a) providing a sample of patient blood;

(b) extracting from the blood sample the patient's DNA,

(c) subjecting the DNA to hybridization with primers specific for any sequence coding for lithium-sodium countertransporter;

(d) polymerizing said sequences, to give polymerized sequences;

(e) amplifying said polymerized sequences, to give an amplified sample of patient sequences;

(f) subjecting the amplified sample to in vitro membrane-based translation to give a translated sample within a cell; and

(g) subjecting the translated sample to flux analysis of lithium, to evaluate sensitivity to lithium therapy in manic depressive patients.

Specifically, the first and second methods are drawn to sequences to any lithium-sodium countertransporter selected from the group consisting of hPiT-1, HPiT-2, and hBNP1, said sequences identified as SEQ.ID.NO.: 1, SEQ.ID.NO.: 2, SEQ.ID.NO.: 3, SEQ.ID.NO.: 4, SEQ.ID.NO.: 5, and SEQ.ID.NO.: 6.

In another embodiment of the present invention, there is provided a third method of evaluating sensitivity to lithium therapy in manic depressive patients, comprising the steps of

(a) providing a sample of patient blood;

(b) isolating the erythrocytes;

(c) subjecting the erythrocytes to flux analysis of lithium, to evaluate sensitivity to lithium therapy in manic depressive patients.

In another embodiment of the present invention, there is provided a fourth method of evaluating lithium-sodium countertransport in patients with mental illness, comprising the steps of

(a) providing a sample of patient blood;

(b) isolating the erythrocytes;

(c) subjecting the erythrocytes to flux analysis of lithium, to evaluate lithium-sodium.

Phosphorus is a major dietary element essential to most important biological molecules. Its absorption from the gut and reabsorption from the glomerular filtrate is by secondary active transport on a family of Na—PO₄ cotransporters. The gene product responsible for this function in erythrocytes is pharmacologically distinct from the previously characterized renal brush border Na—PO₄ cotransporter. Both the brain and peripheral nerve forms (PiT-1, PiT-2, BNPI) and the red blood cell form of the Na—PO₄ cotransporter can use Li⁺ as a congener for Na⁺. Also, arsenate is transported by nerve membranes and probably by red blood cells. Therefore, these two tissues most likely have the same cotransporter/receptor. The renal Na—PO₄ cotransporter, presumably the apical isoform, has been cloned from several species. Applicants have identified the erythrocyte isoform as PiT-1.

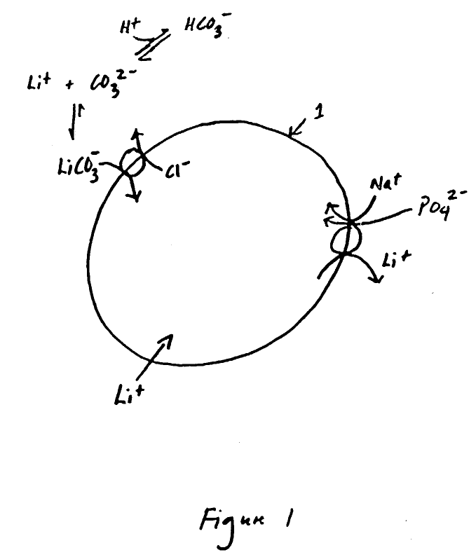

FIG. 1 schematically shows a simplified diagram, with cell 1. Lithium cation, Li⁺ enters the cell as the anion LiCO₃⁻ by the action of the AE1 (Anion Exchange Protein, Band 3) countertransporter. Alternatively, the lithium cation leaks into the cells by a minor unknown leak pathway. It is pumped out by the sodium-phosphate cotransporter, which applicants have identified to be also the lithium-sodium countertransporter.

1. Manipulations of DNA for the Preparation of Expression Systems and Other Purposes

Following well known and conventional practice, the hPiT-1gene or other coding sequences for the lithium-sodium countertransporter are prepared for the expression systems and diagnostic assays of the present invention. These polynucleotide sequences are prepared by ligation of other sequences, restriction endonuclease digestion, cloning, mutagenesis, organic synthesis, or combination thereof, in accordance with the principles and practice of constructing DNA sequences. For sequencing DNA, e.g., verification of a construct at the end of a series of steps, dideoxy DNA sequencing is the preferred method. Other DNA sequencing methods are well known.

Many treatises on recombinant methods have been published, including J. Sambrook et al., Molecular Cloning: A Laboratory Manual 1989; L. G. Davis et al., Basic Methods in Molecular Biology Elsevier 1986; F. M. Ausubel, et al (eds.), Current Protocols in Molecular Biology, Wiley Interscience 1994 (loose-leaf). Such methods include plasmid purification, RNA isolation, Northern blots, Southern blots, Western blots, gel electrophoresis, cDNA library construction, DNA sequencing, amplification by the polymerase chain reaction, cell free translation of mRNAs, and ligation.

Phosphoramidite chemistry in solid phase is the preferred method for the organic synthesis of oligodeoxynucleotides and polydeoxynucleotides. Many other organic synthetic methods are available and are readily adapted to the particular sequences of this invention by a person skilled in the art.

Amplification of DNA or cDNA is a common step in the detection of specific sequences in the diagnostic tests of the present invention. It is typically performed by the polymerase chain reaction (PCR). See, e.g., Mullins, K. et al., U.S. Pat. No. 4,800,159 and other published sources. The basic principle of PCR is the exponential replication of a DNA sequence by successive cycles of primer extension. The extension products of one primer, when hybridized to another primer, becomes a template for the synthesis of another nucleic acid molecule. The primer template complexes act as substrate for DNA polymerase which, in performing its replication function, extends the primers. The region in common with both primer extensions, upon denaturation, serves as template for a repeated primer extension. The conventional enzyme for PCR applications is the thermostable DNA polymerase isolated from Thermus aquaticus, or Taq DNA polymerase. Numerous variations in the PCR protocol exist, and a particular procedure of choice in any given step in the constructions of this invention is readily performed by a skilled artisan. For example, primers for hPiT-1 are organically synthesized, based on its known sequence, and are hybridized to a sample of patient DNA. PCR in combination with reverse transcriptase, so-called RT-PCR, is then carried out to amplify the patient hPiT-1 genes. Subsequent analysis, e.g., by restriction fragment length polymorphism (RFLP), provides information on patient status.

2. Translation of mRNA

Various techniques have been developed to synthesize or isolate large quantities of capped eukaryotic mRNAs, and are readily adaptable to mRNA coding for hPiT-1 and related sequences. Preferably the source for mRNA is derived from enzymological manipulations, rather than isolation of naturally transcribed mRNA from, e.g., cell lines such as the erythroleukemic cell line K562. Synthetic capped mRNA is preferably prepared by in vitro transcription of the appropriate linearized cDNA constructs containing the appropriate promoter for an RNA polymerase, e.g., T7 RNA polymerase. See, e.g., Fletcher, L. et al., J. Biol. Chem. 265:19582 (1990), herein incorporated by reference for these purposes. Under these conditions, high yields of capped mRNA coding for hPiT-1, hPiT-2 or BNPI sequences are obtained, which migrate as a discrete band in gel electrophoresis.

The capped mRNA is then subjected to in vitro membrane-based translation, e.g., in Xenopus oocytes, microsomes or cultured cells, in an expression system designed to permit flux analysis of Na, PO₄, and Li. Preferred expression systems include Xenopus oocytes, and transfected HEK 293 cells. Other suitable transfection systems include Dictyostelium discoideum cells, baculovirus-infected ceected Sf9 cells, and CHO cells. Selection of the appropriate cell system, as well as adjusting the experimental parameters to enhance translation, is readily determined within the skill of the art.

3. Construction of Expression Vector.

The gene for the countertransporter proteins, such as the hPiT-1 gene, is also suitable for expression in an expression vector in a recombinant expression system. Of course, the constructed sequence need not be the same as the original, or its complimentary sequence, but instead may be any sequence determined by the degeneracy of the DNA code. Conservative amino acid substitutions may also be employed or other modifications, such as an amino terminal methionine.

A ribosome binding site active in the host expression system is ligated to the 5′ end of the chimeric coding sequence, giving a synthetic gene. The resulting synthetic gene can be inserted into any one of a large variety of vectors for expression, by ligating to an appropriately linearized plasmid. Expression in E. coli is suitable for expression of active lithium-sodium countertransporter protein, e.g., E. coli BL21. A regulatable promoter is also suitable for the expression of these coding sequences, e.g., under the control of the E. coli lac promoter. Other suitable regulatable promoters include trp, tac, recA, T7, lambda promoters.

4. Diagnostic Assays to Measure Lithium-Sodium Countertransport

The flux of a molecule is a measure of the number of molecules that cross the cell membrane per unit time and per unit of membrane (expressed either as area or number of cells or amount of cell protein). The flux is measured by determining the appearance or disappearance (or both) of the molecule on one side of the membrane. The amount on one side of the membrane is measured at different known times either by a chemical determination or by a radioactive determination if a tracer of the atoms or molecules is used.

Lithium, atomic number 3, atomic weight 6.9 Daltons, has no radioactive isotopes of use for biological measurements. Chemical detemination must be used instead of a radioisotope. The amount of lithium is most often determined by atomic absorption spectroscopy or emission spectroscopy. The assay of lithium-sodium countertransport flux rate is made by the following steps:

1) a sample of whole blood, e.g. 10 ml, is taken from the patient by venipunture;

2) the cells are mixed in a standard buffered solution containing sodium and lithium chloride solution, and subjected to repeated suspension, centrifugation, removal of supernatant fluid, and resuspension;

3) the cells in the standard solution are incubated in the presence of inhibitors of the Na, K, ATPase (e.g., ouabain at 10⁻⁵ M) and in the presence of inhbitors of Anion Exchange protein (e.g., denitrostilbenedisulfonate at 2.5×10⁻⁴ M), in suspension at body temperature;

4) at given known times samples of cells are removed, cooled on ice to slow the further transport of lithium, then washed 3 times by centrifugation, aspirated to remove supernatant and resuspended in an ice cold lithium-free solution, to give washed cells;

5) the washed cells are lysed with lithium-free water;

6) aliquots of lysed cells are taken and diluted if necessary to measure hemaglobin [(van Kampen, E. J. et al., Clin. Chim. Acta 6:538 (1961)] and lithium by flame spectroscopy; and

7) the flux equals the change in lithium per g hemoglobin between samples from the same suspension, divided by the time between samples.

5. Genetic Screening Tests

A variety of methods exist for the evaluation and screening of human DNA sequences obtained as patient samples, for the purpose of patient evaluation. See generally, Caskey, C. T., Science 236: 1223 (1987); Bloch, W., Biochemisity 30:2735 (1991); Erlich, H. A. et al., Science 252: 1643 (1991).

In the classic analysis of polynucleotide sequences by the technique of restriction fragment length polymorphism (RFLP), natural variations in DNA are detected by digestion of DNA, whether or not amplified, with a selected set of restriction endonucleases. The polymorphism need not overlap the site of etiological origin to be evaluated and tested, e.g., the PiT-1 gene, but instead may be a neighboring region linked thereto, e.g. linkage disequilibrium. In one modification of RFLP, a single base pair mutation of a DNA coding strand affects its digestion by a selected restriction endonuclease, and its presence is readily detected by the appropriate primers and PCR (polymerase chain reaction). These types of analytical methods are advantageous because there is no need for a hyribidization reaction of target to labeled probe.

In another technique, known as oligonucleotide complementarity, allele-specific oligonucleotides (ASO) are synthesized for a variety of purposes. These oligonucleotides are useful for either directly hybridizing to target DNA under specific stringency conditions, or for priming in vitro amplification by the polymerase chain reaction.

Tagging or labeling the desired polynucleotide fragments can take various forms. The radioisotope ³²P and other radioactive labels are not preferred because of laboratory safety and waste disposal requirements. Alternative methods of labeling include chemical analogs, such as biotinylated analogs of TTP and UTP, which incorporate into the resulting DNA and RNA, respectively. The biotin-labeled probe can be coupled to avidin, or streptavidin, and the complex detected by chemiluminescence, immunofluorescence, immunoperoxidase, immune colloidal gold techniques, or the like. The biotin-labeled probe can also be detected with avidin conjugated to poly AP (calf intestinal alkaline phosphatase), assayed with the appropriate AP substrates. Digoxigenin is a useful substitute for avidin in many applications, and it is readily detected with antibodies specific for digoxigenin. Various combinations of such labels are readily carried out, e.g., a biotin-labeled probe detected with streptavidin conjugated to poly AP, or a biotin labeled probe detected with anti-biotin antibodies linked to AP, or other secondary labeling systems.

6. Preparation of Antibodies Specific for the Lithium-Sodium Countertransporter Protein, and Allelic Variants Thereof.

Monoclonal antibodies are the reagent of choice in the present invention, and a specifcally used to analyze patient cells for specific characteristics of the lithium-sodium countertransporter. Monospecific antibodies to the lithium-sodium countertransporter are purified from mammalian antisera containing antibodies reactive against the lithium-sodium countertransporter or are prepared as monoclonal antibodies reactive with the lithium-sodium countertransporter using the technique of Kohler and Milstein. Nature, 256: 495-497 (1975). Monospecific antibody as used herein is defined as a single antibody species or multiple antibody species with homogenous binding characteristics for the lithium-sodium countertransporter. Homogenous binding as used herein refers to the ability of the antibody species to bind to a specific antigen or epitope, such as those associated with the lithium-sodium countertransporter, as described above. The lithium-sodium countertransporter specific antibodies are raised by immunizing animals such as mice, rats, guinea pigs, rabbits, goats, horses and the like, with an appropriate concentration of the lithium-sodium countertransporter either with or without an immune adjuvant.

Preimmune serum is collected prior to the first immunization. Each animal receives between about 0.1 mg and about 1000 mg of the lithium-sodium countertransporter associated with an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of the lithium-sodium countertransporter in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of the antigen in Freund's incomplete adjuvant by the same route. Booster injections are given at about three week intervals until maximal titers are obtained. At about 7 days after each booster immunization or about weekly after a single immunization, the animals are bled, the serum collected and aliquots are stored at about −20° C.

Monoclonal antibodies (mAb) reactive with the lithium-sodium countertransporter are prepared by immunizing inbred mice, preferably Balb/c, with the lithium-sodium countertransporter. The mice are immunized by the IP or SC route with about 0.1 mg to about 10 mg, preferably about 1 mg, of the lithium-sodium countertransporter in about 0.5 ml buffer or saline incorporated in an equal volume of an acceptable adjuvant, as discussed above. Freund's complete adjuvant is preferred. The mice receive an initial immunization on day 0 and are rested for about 3 to about 30 weeks. Immunized mice are given one or more booster immunizations of about 0.1 to about 10 mg of the lithium-sodium countertransporter in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS1/Ag 4-1; MPC-11; S-194 and Sp 2/0, with Sp 2/0 being preferred. The antibody producing cells and myeloma cells are fused in polyethylene glycol, about 1000 mol. wt., at concentrations from about 30% to about 50%. Fused Hybridoma cells are selected by growth in hypoxanthine, thymidine and aminopterin supplemented Dulbecco's Modified Eagles Medium (DMEM) by procedures known in the art. Supernatant fluids are collected from growth positive wells on about days 14, 18, and 21 and are screened for antibody production by an immunoassay such as solid phase immunoradioassay (SPIRA) using the lithium-sodium countertransporter as the antigen. The culture fluids are also tested in the Ouchterlony precipitation assay to determine the isotype of the mAb. Hybridoma cells from antibody positive wells are cloned by a technique such as the soft agar technique of MacPherson, Soft Agar Techniques, in Tissue Culture Methods and Applications, Kruse and Paterson, Eds., Academic Press, 1973.

Monoclonal antibodies are produced in vivo by injection of pristane primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production in anti-lithium-sodium countertransporter mAb is carried out by growing the hydridoma in DMEM containing about 2% fetal calf serum to obtain sufficient quantities of the specific mAb. The mAb are purified by techniques known in the art.

Antibody titers of ascites or hybridoma culture fluids are determined by various serological or immunological assays which include, buy are not limited to, precipitation, passive agglutination, enzyme-linked immunosorbent antibody (ELISA) technique and radioimmunoassay (RIA) techniques. Similar assays are used to detect the presence of the lithium-sodium countertransporter in body fluids or tissue and cell extracts.

It is readily apparent to those skilled in the art that the above described methods for producing monospecific antibodies may be utilized to produce antibodies specific polypeptide fragments of the lithium-sodium countertransporter, or full-length nascent lithium-sodium countertransporter polypeptide, or variants or alleles thereof

7. Manic Depression and Other Affective Disorders

The classification of mental illness is fluid and subject to further adjustments and refinements. Two distinct types of mental illness are schizophrenic disorders and affective disorders. Schizophrenic disorders are mental diseases with a tendency toward chronicity and are characterized by psychotic symptoms involving disturbances of thinking, feeling, and behavior. Affective disorders, also known as mood disorders, are psychopathologic states in which a disturbance of mood is either a primary determinant or constitutes the core manifestation. A clinically useful division of affective disorders is bipolar (with periods of depression and elevation) and unipolar (depressions only) mood disturbances. Such bipolar mood disturbances arc commonly known as manic depression.

Lithium, usually given as a carbonate salt, attenuates bipolar mood swings, without affecting normal mood. It also appears to be useful in the treatment of aggressive personality disorders, which are typically classified outside of affective disorders. About 50% of bipolar patients respond to lithium therapy. Various clinical attributes are useful in assessing response to lithium, including the presence of manic episodes as the primary mood disorder, an episode frequency of less than about 2 years, as well as past or family history of lithium response. Applicants now provide another attribute to evaluate response to lithium, that is, lithium-sodium countertransport.

8. Lithium Flux Mechanisms

Lithium is commonly used to treat affective disorders. The site of action of lithium is believed to be in the brain. The steady state ratio of intracellular red blood cell lithium concentration to plasma lithium concentration during therapy shows great interindividual variation, although the lithium ratio is relatively constant for any one individual. Individual fluctuations of the lithium ratio have also been reported. The relative constancy of the ratio in an individual may be genetically determined.

The steady state lithium ratio across the red cell membrane is the result of three lithium transport processes: the Na,K,ATPase which is inhibited by ouabain and other cardiac glycosides, the anion exchange protein (AE1, band 3) and the lithium-sodium countertransport system. The Na, K, ATPase pumps Li into the cell by substituting Li⁺ at a normal K⁺ binding site, but at therapeutic levels of Li⁺ (1-2 mM) and normal plasma Na and normal plasma K, the Na,K ATPase carries Li poorly (<<0.025 mmol/lit cell•h; <<75 μmol/kg Hgb•h). In plasma like media with 24 mM bicarbonate, the anion exchanger is the principle mediator of the inward leak of Li as the ion pair LiCO₃⁻. This transport is inhibited by stilbene disulfonates (SITS, DNDS, DIDS), phloretin and dipyridamol. The lithium-sodium countertransporter normally pumps Li out of the cell against its electrochemical gradient so that [Li]_{cell} is lower than [Li]ₚₗ and [Li]_{cell}/[Li]ₚₗ, which lithium ratio is 0.2 to 0.8 in different individuals. A higher steady ratio is expected in alkalosis (higher plasma HCO₃⁻, CO₃²⁻, and LiCO₃⁻) due to increased AE1 mediated leak into the cell, or when the lithium-sodium countertransport extrusion of lithium is slowed, either because cell Na⁺ gradient is decreased or because the countertransporter is less effective.

There is evidence that the differences in the steady state ratio are principally due to differences in the activity of the Na/Li exchanger (lithium sodium countertrnsporter). For example, there is a correlation between the Li influx on the lithium-sodium countertransporter (which is reversible and will run backward given a reversed Na gradient) and the steady state ratio. Also, the steady state Na ratio does not correlate with the steady state Li ratio in different donor cells after 24 hr in vitro. Thus the “tightness” of the Na/Li coupling varies among individuals.

Applicants have identified the lithium-sodium countertransporter as the product of PiT-1 gene previously identified as retrovirus receptor and a NaPO₄ cotransporter. Applicants have shown that the red cell NaPO₄ cotransporter transports Li instead of Na (i.e., LiPO₄ cotransport) and that is performs Na/Na exchange and lithium-sodium countertransport.

The lithium ratio has been implicated in the responsiveness of polar disease to lithium treatment, the development of essential hypertension (hypertension of unknown etiology), the susceptibility of individuals to affective (bipolar) disorders, and the toxic side effects of Li therapy.

### EXAMPLE 1

Kinetic Evidence that the Sodium-Phosphate Cotransporter is the Major Molecular Mechanism for Na—Li Exchange in Human Red Blood Cells.

Lithium influxes, ³²PO₄ influxes, sodium effluxes were measured in human red blood cells incubated in an isotonic media containing (mM): 150(Na+Li+K)Cl, 0.3 K₂HPO₄, 20 HEPES (4-(2-hydroxyethyl)-1-piperazineethanesulfonic acid), 0.25 DNDS (4,4′-dinitro stilbene-2,2′-disulfonate to inhibit the Anion Exchange Protein Band3 pathway for phosphate transport), titrated to pH 7.64 with KOH at 20° C. to give a pH of 7.40 at 37° C. where the fluxes were performed. First, external lithium (in the absence of sodium) activated phosphate influx. Lithium activation of phosphate influx was increased by 1, 7.5 and 75 mM external sodium. Second, external lithium stimulated Na efflux in the presence of 10⁻⁵ M ouabain (an inhibitor of the Na—K pump) and was further stimulated by external phosphate. These results indicate that the majority of sodium efflux is on the sodium-phosphate cotransporter. Third, external phosphate concentrations slightly inhibited lithium influxes at low (0.1-0.3 mM) phosphate concentrations. Fourth, arsenate inhibited sodium-phosphate cotransport in red blood cells with a Ki of 5.2 mM, more than 10 fold greater than in HEK-293 cells, which have a renal ype II sodium-phosphate cotransporter. The collective results indicate that a mechanism for Na—Li exchange on the sodium-phosphate cotransporter. Phosphate is likely to be an important regulator of lithium transport and its therapeutic effects.

### EXAMPLE 2

Kinetic Characterization of Sodium-Phosphate Cotransporter in the Erythroleukemic Cell Line K562: Identification of the Erythrocyte Sodium Phosphate Cotransporter as hPiT-1.

Na-dependent ³²PO₄ influx into the erythroleukemic line of K562 cells was measured. The ³²PO₄ influx was linear with time of over 30 minutes and was activated over 100-fold by 140 mM Na compared to isomolar substitution by 140 MM N-methyl-D-glucamine. The activation of ³²PO₄ influx by extracellular phosphate (pH 7.4 at 37° C.) was hyperbolic with a Kₘ^{PO4}=0.36 mM and Vₘₐₓ=4500 nmol PO₄/g protein−min in 140 mM Na⁴. The K_{1/2}^{Na}=40 mM when PO₄₀ was 0.3 mM. There was no activation of phosphate influx by Rb, K, or Cs. However, 140 mM Li activated phosphate influx to 18.7% of that realized in the presence of 140 MM Na. The K₁ value for arsenate inhibition of Na-dependent ³²PO₄ influx K562 cells was 2.6 mM. These and other kinetic characteristics of sodium-phosphate cotransport in K562 cells are identical to those previously described for human erythrocytes. See Shoemaker et. al., J. Gen. Physiol, 92:449 (1988).

### EXAMPLE 3

Molecular Identification of the Sodium-Phosphate Cotransporter in Erythroleukemic Cell Line K562 and Erythrocytes as hPiT-1.

Human PiT-1 (hPiT-1) was cloned as the human isoform of the gibbon ape retrovirus receptor [Van Zeijl, M., et al (Proc. Nat. Acad. Sci. 91:1168 (1994)]. PCR primers were designed to amplify either the hPiT-1 or hPiT-2 isoforms. The 1700 bp product was amplified by RT-PCR from total RNA isolated from K562 cells, and restriction analysis with SphI identifies the product as being derived from hPiT-1 and not hPiT-2. This evidence, considered together with the kinetic evidence of preceeding Example, indicates that hPiT-1 is the sodium-phosphate cotransporter isoform present in both K562 cells and erythrocytes.

RT-PCR was carried out using 1 μg of total RNA isolated from K562 cells using the method of Chomczynski et al., Analytical Biochem. 162: 156 (1987). The RT-PCR reaction was carried out in a single tube using recombinant Tth DNA-polymerase which is capable of reverse transcriptase activity under appropriate reaction condition. The primers used in these experiments (F1 and R1) are based upon highly conserved regions between hPiT1 and hPiT2 located in putative transmembrane domains in the N-terminal and C-terminal regions of the proteins. The results from these experiments show that the products of SphI digestion are 1000, 487 and 138 bp. which agree with the predicted sizes. The gel patterns under the PiT-1 control after SphI digestion are the same as for K562, after SphI digestion and both are different from those predicted for PiT-2 after SphI digestion. Thus K562 cells have the human PIT-1 isoform.

### EXAMPLE 4

Synthesis of mRNA for Cell-Free Translation and Xenopus Oocyte-Injection Experiments.

The template for expression of the Na—PO₄ cotransporter in the cell-free system or in Xenopus oocytes is capped mRNA prepared polymerase, prepared by in vitro transcription of linearized cDNA constructs containing the promoter for T7 RNA polymerase. Samples of mRNAs are prepared as prepared as previously described in a 1 ml reaction mixture [Fletcher, L. et al., J. Biol. Chem. 265;19582(1990)]. These reaction conditions permit high yields of mRNA (˜1000 mg or about 100-200 copies of RNA transcript per copy of template DNA) that are >80% capped and migrate as a single discrete band of the correct molecular weight on denaturing PAGE. T7 RNA polymerase is purified as described by Davanloo, et al. Proc. Natl. Acad. Sci. 81;2035(1984).

### EXAMPLE 5

Cell-Free Expression of Na—PO₄ Cotransporter

The Na—PO₄ cotransporter mRNA is expressed in a wheat germ cell-free system containing in vitro transcribed mRNA, dog pancreatic microsomes and signal recognition particle (SRP). Cell-free translation is carried out in the presence of 34 mM [¹⁴C]leucine (50 cpm/pmol), 50 mM each of the other 19 amino acids, unfractionated wheat germ tRNA and other components a previously described [Erickson, A. H et al., Methods Enzymol 96;38 (1983)].

Assuming each Na—PO₄ cotransporter polypeptide contains˜75 leucine (hPiT-1 has 71), synthesis under these conditions yields Na—PO₄ cotransporter with a specific activity of ˜3750 cpm/pmol Wheat germ extract is prepared as described by Lax. S. R. et al. Methods Enzymol. 118;109 (1986), and dog pancreatic microsomes and SRP arc prepared according to Walter, P. et al., Methods Enzymol. 96; 84-93, & 682-691(1983). Under the appropriate conditions the wheat germ system provides excellent activity, e.g. 5 5 mol β-globin polypeptide is synthesized per mol β-globin mRNA template per hour. These data indicate that a 1 ml reaction containing 100 pmol mRNA synthesizes up to 50 mg Na—PO₄ cotransporter protein (or nearly 500 pmol) per hour. The synthesis of Na—PO₄ cotransporter is monitored by SDS-PAGE and fluorography to determine that the correct molecular weight polypeptide is produced. Translocation and glycosylation is assessed by endogycosidase H and endoglycosidase F treatment. Treated and untreated samples are analyzed by SDS-PAGE.

### EXAMPLE 6

Heterologous Expression of the Na—PO₄ Cotransporter

The expression of the the sodium-phosphate cotransporter is carried out in several heterologous expression systems, e.g. Dictyostelium discoideum cells, Xeonpus oocytes, HEK 293 cells, baculovirus-infected Sf9 cells, and CHO cells. Transfection, growth and selection of transformants are performed by well known techniques. The expression of the Na—PO₄ cotransporter is assessed by immunoprecipitation with Na—PO₄— specific antibodies of the protein from cells (Dictyostelium, HEK 293, etc.) grown in medium supplemented with ³⁵S-methionine. The functional expression of the Na—PO₄ transporter, both native and mutant forms, in transfected cells is monitored by determining the Na-dependent ³²Na—PO₄ flux, as described in an Example below. Negative controls include determining background levels of Na-dependent Na—PO₄ transport from cells transfected with the construct in the anti-sense orientation. The two principal expression systems for heterologous expression of the erythrocyte Na—PO₄ cotransporter are injected Xenopus oocytes and transfected Dictostelium. Alternatively or additionally, the cotransporter is expressed in another expression system such as HEK 293 or baculovirus-infected Sf9 cells.

A. Injected Xenopus oocytes. Stage V and IV oocytes are removed using standard anesthetic (0.17% 3-aminobenzoic acid) and surgical procedures. The oocytes are placed in OR-2 medium and collagenase treated (2 mg/ml) for 2.5 h. Individual oocytes are washed and defolliculated if needed by trituration and co-injected with 2.5 ng capped SEAP cRNA and 5-50 ng of capped transporter of cRNA (prepared as described above). Capped SEAP cRNA prepared by in vitro transcription of HIndIII linearized pGEM-SEAP. The pGEM-SEAP construct contains the human placental alkaline phosphatase with a site-specific mutation at codon 489 to create a termination codon[Tate, S. S. et al., FASEB J. 4; 228 (1990)]. This stop codon results in a secreted form of alkaline phosphatase rather than a membrane anchored form. cRNAs are injected in a total volume of 50 nl using a Narishige injector. Following incubation overnight in Barth's medium, oocytes are sorted and placed in single wells of a 96 well plate containing 200 ul Barth's medium. Five hours after the oocytes are placed into individual wells, 50 ul of medium is removed for SEAP activity assay. Alkaline phosphatase activity is measured by chromogenic assay. The secreted alkaline phosphatase catalyzes the dephosporylation of nicotinamide adenine dinucleotide phosphate (NADP⁺); the NAD formed then catalytically activates an NAD⁺-specific oxidation-reduction cycle driven by the enzymes alcohol dehydrogenase and diaphorase. The chromophore formed is a violet colored formazan product of INT-violet. Only those oocytes that express SEAP (10 units activity/50 ul at 29 h post-injection) are used in the flux measurements. There is substantial correlation between the level of SEAP activity detected at 29 h post-injection and the level of ³⁶Cl flux in oocytes co-injected with SEAP and human AE1 or the level of Na-activated ³²PO₄ influx in oocytes injected with SEAP and mRNA for PiT-1, PiT-2, or BNTPI. Those oocytes that are positive for SEAP expression are incubated for an additional 1-5 days with daily changes in Barth's medium before influx assays are carried out.

B. Dictyostelium. A second expression system for heterologous expression of the cloned cotransporter is Dictyostelium. A significant advantage of the Dictyostelium expression system is that these cells are grown in suspension culture and are handled like red cells for flux measurements. The principle expression vector (e.g. pBS18 and its derivatives) is based upon selection using the Tn5 gene (neomycin phosphotransferase II) driven by the actin 6 promoter. The insert of interest is driven by the actin 8 promoter and the 2H3 transcriptional terminator. The Tn5 gene permits selection of permanent transfectants in media containing G-418, to which the native slime mold is highly sensitive.

C. HEK-239 cells. Heterologus expression is also readily carried out with HEK-293 cells, ATCC Accession No. CRL 1573. HEK-293 (human embryonic kidney) cells were obtained from the American Type Culture Collection (ATCC) at passage 31 These cells were used to prepare seed stocks at passage 32. Cells were used until passage 45, after which fresh cultures were started from frozen passage 32 cells. The cells were grown in Minimal Essential Media (MEM) with Hank's salts and supplemented with L-glutamine and 5% fetal calf serum at 37° C. in 5% CO₂/95% air. Transfection was carried out using standard calcium phosphate precipitation methods. Specifically, five days before the transfection, cells were plated in T75 flasks (75 cm²) at 2.5×10⁴/cm². On the day of the transfection, the cell density was usually 2-3×10⁵/cm². The cells were washed and fresh media (20 mL) was placed in each culture flask. A 1.0 mL suspension containing the calcium phosphate—DNA precipitate from 40 μg of plasmid DNA was added drop-wise with mixing to the media overlaying the cells The cells were returned to the incubator for 4 h. then 2 mL of 18% (v/v) glycerol was added to the media (“glycerol shocked”), and the cells incubated for an additional two minutes at room temperature. The media was then quickly aspirated from the flask, the cells washed one time with 25 mL Dulbecco's phosphate-buffered saline, fresh media added to the cells (25 mL) and the cells were incubated overnight. The next morning the cells were trypsinized by standard methods, resuspended to a final density of 1.5-1.7×10⁵ and 1.0 mL of the cell suspension was used to replate the cells in 24-well plates (16 mm diameter wells) at a density of 8.0×10⁴ cells/cm² (1.6×10⁵ cells/well). Flux measurements were carried out at 48±6 hr post transfection.

### EXAMPLE 7

Flux Measurements

A. Flux measurements in cell-free expression system. The flux is measured by an adaptation of a rapid filtration method according to Macintyre, J. D. et al. Biochim. Biophys. Acta 644; 351 (1981). Briefly, microsomes are suspended, equilibrated and mixed in media containing either sodium or choline or N-methyl-alpha-glucamine as the dominant cation in a thermostatically controlled chamber. The flux is initiated by additional of ³²PO₄ to the flux medium. Aliquots are removed at different times and filtered under vacuum using prewashed mixed cellulose-ester filters. The microsomes retained by the filter are rapidly washed with stopping solution containing 323 mM MgSO₄ (isotonic for microsomes). A sample of flux suspension is used to measure total protein and specific activity. The flux (pmol PO₄/ug protein-min or ions/cotransporter molecule-min) is calculated from the slope of cpm/aliquot versus time. Preloaded microsomes are used to verify the quantitative recovery of microsomes, the replication of sample counts, and the effectiveness of the wash in removing a extracellular marker, usually ¹⁴C-PEG at 0° C. The probable ³²PO₄ influxes into microsomes are calculated assuming a single copy of the Na—PO₄ cotransporter in each 0.05 urn microsome, and using kinetic data from erythrocytes, assuming that there are 450 copies of the cotransporter per red blood cell. These calculations indicate that the half-times will be>10 h and are therefore measured by this technique (t_{1/2}>5 sec).

B. Flux measurements in oocytes. Oocytes are prepared and injected as described in Example above. Briefly, eight to ten oocytes are placed in individual wells of a 96-well culture plate in medium containing either Na or choline as the dominant cation. The flux is initiated by addition of ³²PO₄ to each well. As known times, oocytes are removed and washed three times in ice cold choline medium. The oocyte is then dissolved in 0.2 ml 10% SDS and the counted in a liquid scintillation counter. A sample of the ³²PO₄ incubation fluid is counted to calculate the extracellular specific activity. The influx (pmol/oocyte/hr) is calculated from the specific activity and the uptake. The difference between the flux in Na and choline media is the calculated Na-dependent phosphate influx.

C. Flux measurements in Dictyostelium discoideum. HL-5 medium contains 20±3 mM K. This is not a defined medium so the composition must be determined for each flux. Cells are grown to a density of 1-3×10⁶/ml at 20° C. in a shaking incubator. Approximately 10⁷ cells are required for each data point on the influx curve. The cells are resuspended to 4×10⁷/ml in HL-5 in a thermostatted stirred chamber at a known pH. At time zero tracer (˜0.6 μCi/ml) is added and at known times thereafter samples (0.4 ml) are removed. The samples are transferred to 7 ml of ice-cold stop solution (58 mM MgSO₄), immediately centrifuged for 30 seconds at 3000×g in a rotor, the supernatant aspirated and discarded as radioactive waste. The pellet of cells is resuspended thrice in 6 ml of stop solution, pelleted, and the supernatant aspirated To the drained pellets 1 ml of 0.1% DOC in 1 N NaOH is added for solubilization Aliquots are counted for radioactivity or are assayed for protein. The data are calculated as pmoles/mg protein and the data vs. time are fitted to a single exponential by nonlinear regression analysis and the inital slope (flux) and asymptote value constant (pmoles/mg protein) calculated.

D. Flux measurements in cultured mammaliam cells. Human embryonic kidney cells (HEK-293) are purchased from the American Type Culture Collection (ATCC, accession number CRL 1573) at passage 32 and grown in MEM and 5% fetal calf serum in a 5% CO₂/95% air 37° C. incubator. They are maintained in T75 flasks and split weekly. At passage 45 decreased expression is observed, so frozen stocks at passage 34 are brought up. The cells are transfected with cDNA harvested from bacteria and purified on an anion exchange column. At 24 hours the cells are plated onto 24-well plates at 3-5×10⁵ cells/cm² and transport measured at 48-72 hr. The expression is low by 96 hr and absent at 5 and 10 days. Usually the medium is aspirated and washed once with 0.5 ml of a Na-free (143 mM N-methyl-D-glucamine Cl) HCO₃-free MEM-like HEPES buffered medium for 3-5 min. Then it is preincubated at 37° C. in room air for 30 minutes. The flux is initiated by adding ³²PO₄ or ²²Na containing media. Cells transfected with the vector only (e.g., pRBG4) are always treated and fluxed in parallel. ³²PO₄ influx in the absence of Na is always measured. All fluxes are performed in duplicate. The plates are placed on a water thermostatted table for 5 minutes and the flux initiated by aspirating the preincubation medium from the last column of wells and adding the tracer solution at known times (±0.2 sec.). This is done to successive columns of cells at approximately 30 minutes, 15 minutes, 10 minutes, 5 minutes, 2 minutes and 1 minute prior to terminating the influx simultaneously for all wells on the plate by 3 rapid ice cold washes (over 15 seconds total elapsed time) with (mM): 150 NaCl, 1.5 CaCl₂, 1 MgCl₂ solution. Residual wash solution is aspirated and the cells in the dry wells are solubilized in 0.5 ml of 25 mM NaOH with 0.5% deoxycholate. A 50 μl sample from each well is used to measure protein and 400 μl sample is counted in a liquid scintillation counter. Quadruplicate 10 μl samples of the influx solution are counted contemporaneously with the flux samples for specific activity determination. The pmol/μg protein in each well is calculated and the slope of the linear least squares best fit to these values against sample times is the computed flux, according to Sarkadi, B. et al., J. Gen. Physiol. 72: 249 (1978). Usually 5 or 6 of the data points are used to calculate each flux. Each condition is always measured in duplicate in both vector-only transfected and vector+insert transfected cells.

### EXAMPLE 8

Isolation, Cloning, and Sequencing of Llithium-Sodium Countertransporters.

The hPiT-1 DNA was isolated, cloned and sequenced according to U.S. Pat. No. 5,414,076, herein incorporated by reference for this purpose. It is set forth as SEQUENCE ID NO.:1 and SEQUENCE ID NO.:2.

The hPiT-2 DNA was isolated, cloned and sequenced according to U.S. Pat. No. 5,550,221, herein incorporated by reference for this purpose. It is set forth as SEQUENCE ID NO.:3 and SEQUENCE ID NO.:4.

The BNPI DNA was isolated, cloned and sequenced according to Ni, B et al., J. Neurochem. 66, 2227 (1996), herein incorporated by reference for this purpose. It is set forth as SEQUENCE ID NO.:5 and SEQUENCE ID NO.:6.

### EXAMPLE 9

Preparation of Antibodies Specific for the Erythrocyte Na—PO₄ Cotransporter.

Polyclonal antibodies are prepared according to England, B. J. et al., Biochim.Biophys.Acta 623: 171(1980), and Timmer, R. T. et al., J.Biol.Chem. 268 24863 (1993). Monoclonal antibodies are prepared according to Kohler, G. et al., Nature 256: 495 (1975).

### EXAMPLE 10

Restrcition Length Fragment Polymorphism Analysis

A. Using primers for hPiT-1, for example,

| | |
|---|---|
| CAGTTCAGTC AAGCCGTCAG and | (SEQ ID NO: 7) |
| CCAGCCAACA GACACAACAG, | (SEQ ID NO: 8) |

the hPiT-1 sequence is amplified by PCR and ASO, by the methods of Connor, B. J. et al., Proc.Natl.Acad.Sci. 80: 278 (1983), and Saiki, R. K. et al., Nature 324:163 (1986). Subsequent digestion with TaqI, PvuI, MboI, and SacI restriction endouncleases is performed.

B. Using primers for hPiT-2, for example,

| | |
|---|---|
| ACAACGAGAC GGTGGAGACT and | (SEQ ID NO: 9) |
| TGCGGTGTAG CAGGTGTAAC, | (SEQ ID NO: 10) |

the hPiT-2 sequence is amplified by PCR and ASO, by the methods of Connor, B. J. et al., Proc.Natl.Acad.Sci. 80: 278 (1983), and Saiki, R. K. et al., Nature 324: 163 (1986). Subsequent digestion with TaqI, PvuI, AboI, and SacI restriction endouncleases is performed.

C. Using primers for BNPI, for example,

| | |
|---|---|
| CCTCGCCGCT ACATTATCGC and | (SEQ ID NO: 11) |
| CGAAGCCTCC GCAGTTCATC, | (SEQ ID NO: 12) |

the BNPI sequence is amplified by PCR and ASO, by the methods of Connor, B. J. et al., Proc.Natl.Acad.Sci. 80: 278 (1983), and Saiki, R. K. et al., Nature 324: 163 (1986) Subsequent digestion with TaqI, PvuI, MboI, and SacI restriction endouncleases is performed.

While the foregoing specification teaches the principles of the present invention, with examples provided for the purpose of illustration, it will be understood that the practice of the invention encompasses all of the usual variations, adaptations, modifications or deletions as come within the scope of the following claims and its equivalents.

What is claimed is:

1. A purified DNA molecule coding for a lithium-sodium countertransporter.

2. A purified DNA molecule coding for an amino acid sequence selected from the group consisting of hPiT-1, hPiT-2, and hBNPI, said molecule useful for measuring lithium-sodium countertransport in human cells.

3. The purified DNA molecule of claims 1 or 2, wherein the nucleotide sequence is SEQ.ID.NO.: 2.

4. The purified DNA molecule of claims 1 or 2, said DNA encoding for the amino acid sequence of SEQ.ID.NO.: 1.

5. The purified DNA molecule of claims 1 or 2, wherein the nucleotide sequence is SEQ.ID.NO.: 4.

6. The purified DNA molecule of claims 1 or 2, said DNA encoding for the amino acid sequence of SEQ.ID.NO.: 3.

7. The purified DNA molecule of claims 1 or 2, wherein the nucleotide sequence is SEQ.ID.NO.: 6.

8. The purified DNA molecule of claims 1 or 2, said DNA encoding for the amino acid sequence of SEQ.ID.NO.: 5.

9. A human amphotrophic retrovirus receptor useful as a lithium-sodium countertransporter.

10. The receptor of claim 9, wherein its nucleotide sequence is SEQ.ID.NO. 2.

11. The receptor of claim 9, wherein its amino acid sequence is SEQ.ID.NO.: 1.

12. The receptor of claim 9, wherein its nucleotide sequence is SEQ.ID.NO. 4.

13. The receptor of claim 9, wherein its amino acid sequence is SEQ.ID.NO.: 3.

14. The receptor of claim 9, wherein its nucleotide sequence is SEQ.ID.NO. 6.

15. The receptor of claim 9, wherein its amino acid sequence is SEQ.ID.NO.: 5.

16. A method of evaluating sensitivity to lithium therapy in manic depressive patients, comprising the steps of
(a) providing a sample of patient blood;
(b) extracting from the blood sample the patient's DNA;
(c) subjecting the DNA to hybridization with primers specific for any sequence coding for lithium-sodium countertransporter;
(d) polymerizing said sequences, to give polymerized sequences;
(e) amplifying said polymerized sequences, to give an amplified sample of patient sequences;
(f) digesting the amplified sample with one or more restriction endonucleases suitable for mapping sites on the DNA indicating susceptibility to lithium therapy.

17. A method of evaluating sensitivity to lithium therapy in manic depressive patients, comprising the steps of
(a) providing a sample of patient blood;
(b) extracting from the blood sample the patient's DNA;
(c) subjecting the DNA to hybridization with primers specific for any sequence coding for lithium-sodium countertransporter;
(d) polymerizing said sequences, to give polymerized sequences;
(e) amplifying said polymerized sequences, to give an amplified sample of patient sequences;
(f) subjecting the amplified sample to in vitro membrane-based translation to give a translated sample within a cell; and
(g) subjecting the translated sample to flux analysis of lithium, to evaluate sensitivity to lithium therapy in manic depressive patients.

18. The method of claims 16 or 17, wherein the sequence coding for the lithium-sodium countertransporter is selected from the group consisting of hPiT-1, hPiT-2, and hBNPI.

19. The method of claims 16 or 17, wherein the sequence is the nucleotide sequence coding for the lithium-sodium countertransporter selected from the group consisting of SEQ.ID.NO.:2, SEQ.ID.NO.:4,. and SEQ.ID.NO.:6.

20. The method of claims 16 or 17, wherein the sequence is the amino acid sequence for the lithium-sodium countertransporter is selected from the group consisting of SEQ.ID.NO.:1, SEQ.ID.NO.:3,. SEQ.ID.NO.:5.

21. A method of evaluating sensitivity to lithium therapy in manic depressive patients, comprising the steps of
(a) providing a sample of patient blood;
(b) isolating the erythrocytes;
(c) subjecting the erythrocytes to flux analysis of lithium, to evaluate sensitivity to lithium therapy in manic depressive patients.

22. A method of evaluating lithium-sodium countertransport in patients with mental illness, comprising the steps of
(a) providing a sample of patient blood;
(b) isolating the erythrocytes;
(c) subjecting the erythrocytes to flux analysis of lithium, to evaluate lithium-sodium countertransport.


SEQUENCE LISTING (12 sequences)

SEQ ID NO: 1 - protein, 679 aa, Homo sapiens
  hPIT-1 1..679: /label= Receptor1 /note= “/organism=Homosapiens/cell_line=HL60 /tissue_lib=lambda HGR6, 7, and 16; Clontech #1020b/map=2q11-q14”
  hypothetical: NO
  anti-sense: NO
  fragment type: N-terminal
  map position: 2q11-q14
  document number: US 5,414,076 P
  document number: O′Hara, B., et al.,“Characterization of Human Gene Conferring Sensitivity to Infection by Gibbon Ape Leukemia Virus,” Cell Growth Differ. 1(
MATLITSTTAATAASGPLVDYLWMLILGFIIAFVLAFSVGANDVANSFGTAVGSGVVTLKQACILASIFETVGSVLLGAKVSETIRKGLIDVEMYNSTQGLLMAGSVSAMFGSAVWQLVASFLKLPISGTHCIVGATIGFSLVAKGQEGVKWSELIKIVMSWFVSPLLSGIMSGILFFLVRAFILHKADPVPNGLRALPVFYACTVGINLFSIMYTGAPLLGFDKLPLWGTILISVGCAVFCALIVWFFVCPRMKRKIEREIKCSPSESPLMEKKNSLKEDHEETKLSVGDIENKHPVSEVGPATVPLQAVVEERTVSFKLGDLEEAPERERLPSVDLKEETSIDSTVNGAVQLPNGNLVQFSQAVSNQINSSGHSQYHTVHKDSGLYKELLHKLHLAKVGDCMGDSGDKPLRRNNSYTSYTMAICGMPLDSFRAKEGEQKGEEMEKLTWPNADSKKRIRMDSYTSYCNAVSDLHSASEIDMSVKAAMGLGDRKGSNGSLEEWYDQDKPEVSLLFQFLQILTACFGSFAHGGNDVSNAIGPLVALYLVYDTGDVSSKVATPIWLLLYGGVGICVGLWVWGRRVIQTMGKDLTPITPSSGFSIELASALTVVIASNIGLPISTTHCKVGSVVSVGWLRSKKAVDWRLFRNIFMAWFVTVPISGVISAAIMAIFRYVILRM

SEQ ID NO: 2 - RNA (mRNA), 3220 nt; single-stranded, linear
  hPit-1 1..3220: /product= “Leukemia Virus Receptor 1”
  hypothetical: NO
  anti-sense: NO
  document number: US 5,414,076 P
gagctgtccccggtgccgccgacccgggccgtgccgtgtgcccgtggctccagccgctgccgcctcgatctcctcgtctcccgctccgccctcccttttccctggatgaacttgcgtcctttctcttctccgccatggaattctgctccgtgcttttagccctcctgagccaaagaaaccccagacaacagatgcccatacgcagcgtatagcagtaactccccagctcggtttctgtgccgtagtttacagtatttaattttatataatatatattatttattatagcatttttgatacctcatattctgtttacacatcttgaaaggcgctcagtagttctcttactaaacaaccactactccagagaatggcaacgctgattaccagtactacagctgctaccgccgcttctggtcctttggtggactacctatggatgctcatcctgggcttcattattgcatttgtcttggcattctccgtgggagccaatgatgtagcaaattcttttggtacagctgtgggctcaggtgtagtgaccctgaagcaagcctgcatcctagctagcatctttgaaacagtgggctctgtcttactgggggccaaagtgagcgaaaccatccggaagggcttgattgacgtggagatgtacaactcgactcaagggctactgatggccggctcagtcagtgctatgtttggttctgctgtgtggcaactcgtggcttcgtttttgaagctccctatttctggaacccattgtattgttggtgcaactattggtttctccctcgtggcaaaggggcaggagggtgtcaagtggtctgaactgataaaaattgtgatgtcttggttcgtgtccccactgctttctggaattatgtctggaattttattcttcctggttcgtgcattcatcctccataaggcagatccagttcctaatggtttgcgagctttgccagttttctatgcctgcacagttggaataaacctcttttccatcatgtatactggagcaccgttgctgggctttgacaaacttcctctgtggggtaccatcctcatctcggtgggatgtgcagttttctgtgcccttatcgtctggttctttgtatgtcccaggatgaagagaaaaattgaacgagaaataaagtgtagtccttctgaaagccccttaatggaaaaaaagaatagcttgaaagaagaccatgaagaaacaaagttgtctgttggtgatattgaaaacaagcatcctgtttctgaggtagggcctgccactgtgcccctccaggctgtggtggaggagagaacagtctcattcaaacttggagatttggaggaagctccagagagagagaggcttcccagcgtggacttgaaagaggaaaccagcatagatagcaccgtgaatggtgcagtgcagttgcctaatgggaaccttgtccagttcagtcaagccgtcagcaaccaaataaactccagtggccactcccagtatcacaccgtgcataaggattccggcctgtacaaagagctactccataaattacatcttgccaaggtgggagattgcatgggagactccggtgacaaacccttaaggcgcaataatagctatacttcctataccatggcaatatgtggcatgcctctggattcattccgtgccaaagaaggtgaacagaagggcgaagaaatggagaagctgacatggcctaatgcagactccaagaagcgaattcgaatggacagttacaccagttactgcaatgctgtgtctgaccttcactcagcatctgagatagacatgagtgtcaaggcagcgatgggtctaggtgacagaaaaggaagtaatggctctctagaagaatggtatgaccaggataagcctgaagtctctctcctcttccagttcctgcagatccttacagcctgctttgggtcattcgcccatggtggcaatgacgtaagcaatgccattgggcctctggttgctttatatttggtttatgacacaggagatgtttcttcaaaagtggcaacaccaatatggcttctactctatggtggtgttggtatctgtgttggtctgtgggtttggggaagaagagttatccagaccatggggaaggatctgacaccgatcacaccctctagtggcttcagtattgaactggcatctgccctcactgtggtgattgcatcaaatattggccttcccatcagtacaacacattgtaaagtgggctctgttgtgtctgttggctggctccggtccaagaaggctgttgactggcgtctctttcgtaacatttttatggcctggtttgtcacagtccccatttctggagttatcagtgctgccatcatggcaatcttcagatatgtcatcctcagaatgtgaagctgtttgagattaaaatttgtgtcaatgtttgggaccatcttaggtattcctgctcccctgaagaatgattacagtgttaacagaagactgacaagagtctttttatttgggagcagaggagggaagtgttacttgtgctataactgcttttgtgctaaatatgaattgtctcaaaattagctgtgtaaaatagcccgggttccactggctcctgctgaggtcccctttccttctgggctgtgaattcctgtacatatttctctactttttgtatcaggcttcaattccattatgttttaatgttgtctctgaagatgacttgtgatttttttttcttttttttaaaccatgaagagccgtttgacagagcatgctctgcgttgttggtttcaccagcttctgccctcacatgcacagggatttaacaacaaaaatataactacaacttcccttgtagtctcttatataagtagagtccttggtactctgccctcctgtcagtagtggcaggatctattggcatattcgggagcttcttagagggatgaggttctttgaacacagtgaaaatttaaattagtaacttttttgcaagcagtttattgactgttattgctaagaagaagtaagaaagaaaaagcctgttggcaatcttggttatttctttaagatttctggcagtgtgggatggatgaatgaagtggaatgtgaactttgggcaagttaaatgggacagccttccatgttcatttgtctacctcttaactgaataaaaaagcctacagtttttagaaaaaacccgaattc

SEQ ID NO: 3 - protein, 652 aa
  hPiT-2 1..652: /label= Receptor2 /note= “/organism=homo sapiens /sex=male
  hypothetical: NO
  anti-sense: NO
  fragment type: N-terminal
  original source tissue type: <Unknown>
  document number: van Zeijl, M., et al., ”A Human Amphotropic Retrovirus Rece
  document number: US 5,550,221 P
MAMDEYLWMVILGFIIAFILAFSVGANDVANSFGTAVGSGVVTLRQACILASIFETTGSVLLGAKVGETIRKGIIDVNLYNETVETLMAGEVSAMVGSAVWQLIASFLRLPISGTHCIVGSTIGFSLVAIGTKGVQWMELVKIVASWFISPLLSGFMSGLLFVLIRIFILKKEDPVPNGLRALPVFYAATIAINVFSIMYTGAPVLGLVLPMWAIALISFGVALLFAFFVWLFVCPWMRRKITGKLQKEGALSRVSDESLSKVQEAESPVFKELPGAKANDDSTIPLTGAAGETLGTSEGTSAGSHPRAAYGRALSMTHGSVKSPISNGTFGFDGHTRSDGHVYHTVHKDSGLYKDLLHKIHIDRGPEEKPAQESNYRLLRRNNSYTCYTAAICGLPVHATFRAADSSAPEDSEKLVGDTVSYSKKRLRYDSYSSYCNAVAEAEIEAEEGGVEMKLASELADPDQPREDPAEEEKEEKDAPEVHLLFHFLQVLTACFGSFAHGGNDVSNAIGPLVALWLIYKQGGVTQEAATPVWLLFYGGVGICTGLWVWGRRVIQTMGKDLTPITPSSGFTIELASAFTVVIASNIGLPVSTTHCKVGSVVAVGWIRSRKAVDWRLFRNIFVAWFVTVPVAGLFSAAVMALLMYGILPYV

SEQ ID NO: 4 - RNA (mRNA), 3175 nt; single-stranded, linear
  hPiT-2 1..3175: /product= “Leukemia virus receptor 2” /label= Receptor2
  hypothetical: NO
  anti-sense: NO
  original source tissue type: placenta
  document number: van Zeijl, M., et al., “A Human Amphotropic Retrovirus Rece
  document number: US 5,550,221 P
cagatcgggaagaaaaatatggaatgtgttttaccgctgactgaacacaaccaaatgaactgtcctgacagtagtttgcaaaccagcagctagcagtttgtccagcctctaacattgtccagcactttccagagcaaactcactgtttacaagaactcttggccttacgaagtttataacctcaagctttgtttatttaaaatattcctgcaaaagaaaagtacccggcacccactttccaaaatggccatggatgagtatttgtggatggtcattttgggtttcatcatagctttcatcttggccttttctgttggtgcaaacgatgttgccaactcctttggtacagccgtgggctctggtgtggtgaccttgaggcaggcatgcattttagcttcaatatttgaaaccaccggctccgtgttactaggcgccaaagtaggagaaaccattcgcaaaggtatcattgacgtgaacctgtacaacgagacggtggagactctcatggctggggaagttagtgccatggttggttccgctgtgtggcagctgattgcttccttcctgaggcttccaatctcaggaacgcactgcattgtgggttctactataggattctcactggtcgcaatcggtaccaaaggtgtgcagtggatggagcttgtcaagattgttgcttcttggtttatatctccactgttgtctggtttcatgtctggcctgctgtttgtactcatcagaattttcatcttaaaaaaggaagaccctgttcccaatggcctccgggcactcccagtattctatgctgctaccatagcaatcaatgtcttttccatcatgtacacaggagcaccagtgctcggccttgttctccccatgtgggccatagccctcatttcctttggtgtcgccctcctgttcgctttttttgtgtggctcttcgtgtgtccgtggatgcggaggaaaataacaggcaaattacaaaaagaaggtgctttatcacgagtatctgacgaaagcctcagtaaggttcaggaagcagagtccccagtatttaaagagctaccaggtgccaaggctaatgatgacagcaccatcccgctcacgggagcagcaggggagacactggggacctcggaaggcacttctgcgggcagccaccctcgggctgcatacggaagagcactgtccatgacccatggctctgtgaaatcgcccatctccaacggcaccttcggcttcgacggccacaccaggagcgacggtcatgtgtaccacaccgtgcacaaagactcggggctctacaaagatctgctgcacaaaatccacatcgacaggggccccgaggagaagccagcccaggaaagcaactaccggctgctccgccgaaacaacagttacacctgctacaccgcagccatttgtgggctgccagtgcacgccacctttcgagctgcggactcatcggccccagaggacagtgagaagctggtgggcgacaccgtgtcctactccaagaagaggctgcgctacgacagctactcgagctactgtaacgcggtggcagaggcggagatcgaggcggaggagggcggcgtggagatgaagctggcgtcggagctggccgaccctgaccagccgcgagaggaccctgcagaggaggagaaggaggagaaggacgcacccgaggttcacctcctgttccatttcctgcaggtcctcaccgcctgtttcgggtcctttgctcacggcggcaatgacgtgagtaatgccatcggtcccctggtagccttgtggctgatttacaaacaaggcggggtaacgcaagaagcagctacacccgtctggctgctgttttatggaggagttggaatctgcacaggcctctgggtctgggggagaagagtgatccagaccatggggaaggacctcactcccatcacgccgtccagcggcttcacgatcgagctggcctcagccttcacagtggtgatcgcctccaacatcgggcttccagtcagcaccacgcactgtaaggtgggctcggtggtggccgtgggctggatccgctcccgcaaggctgtggactggcgcctctttcggaacatcttcgtggcctggttcgtgaccgtccctgtggctgggctgttcagcgctgctgtcatggctcttctcatgtatgggatccttccatatgtgtgatttgtcttcttccagctgcaaacagctaaagggatggtctggtgttggcgtgtgggagacatgtgtgctcgtgccgcacatacacatcctggccgtgcacggctctctcatgaccagctctctgcctcccttccaggaggctccatcccacactgttcacccaggctgcggagactcaccttcccgagctaacttaactactgtacataataatatgtattaaactggtatcgtggtgatataatgtggtgcagttacttatatattaaatatctattgtatccatagaataggcagcattatttcaaacatattcaagttgggagtggagatcattgcctagaagtcaatattcaataaatcttgtacataactatttcgatggcaaatgttaagccttctaaaaggaaagtgtagattggaaaatgattttttttccaaatgatgtttttgccttctaatatactgtaaggtaatgagcttcagaacaggcaacctgaccctgcagaggtcgcgtgctgtgggatgacagcgggacgggagctcacaagtgctttcactgaagatttgttcatatactgtgtattgattgttgtgtaatatatcatcattgcttttgtaaatacgtaaaactgtaattttttaatggtgtgcttcccttatactttttgatcagagaattttggaaagtaccaaagaagcaggggaatcattggccagtgttacgttttcacattgtctgtctcccaccctcactgatcacgcctgccccagagcagtgtgtggcggtgacaccgtcacccagcatgcgccacgccgtcgtcccaccagcagtgccaccgccaccacaccccagatcccacccaccttgcagtggctttcttgtcatcagagtagagaatgcacaggtgttggtgagggcgtgtggctgagcactacatgtcaagtcagagtcagtttctatccaattctc

SEQ ID NO: 5 - protein, 560 aa
  hBNPI 1..560
  hypothetical: NO
  anti-sense: NO
  fragment type: N-terminal
  document number: Ni, B., et al., J. Neurochem., 66:2227 (1996)
MEFRQEEFRKLAGRALGKLHRLLEKRQEGAETVELSADGRPVTTQTRDPPVVDCTCFGLPRRYIIAIMSGLGFCISFGIRCNLGVAIVSMVNNSTTHRGGHVVVQKAQFSWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPLTKFSTPWRRFFTSMPVYAIIVANFCRSWTFYLLLISQPDYFEEVFGFEISKVGLVSALPHLVMTIIVPIGGQIADFLRSRRIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDDSEMEDEAEPPGAPPAPPPSYGATHSTFQPPRPPPPVRDY

SEQ ID NO: 6 - RNA (mRNA), 2716 nt; single-stranded, linear
  hBNPI 1..2716
  hypothetical: NO
  anti-sense: NO
  document number: Ni, B., et al., J. Neurochem., 66:2227 (1996)
cgataagcttgatatcgaattccggactcttgctcgggcgccttaacccggcgttcggttcatcccgcagcgccagttctgcttaccaaaagtggcccactaggcactcgcattccacgcccggctccacgccagcgagccgggcttcttacccatttaaagtttgagaataggttgagatcgtttcggccccaagacctctaatcattcgctttaccggataaaactgcgtggcgggggtgcgtcgggtctgcgagagcgccagctatcctgagggaaacttcggagggaaccagctactagatggttcgattagtctttcgcccctatacccaggtcggacgaccgatttgcacgtcaggaccgctacggacctccaccagagtttcctctggcttcgccctgcccaggcgatcggcgggggggacccgcggggtgaccggcggcaggagccgccaccatggagttccgccaggaggagtttcggaagctagcgggtcgtgctctcgggaagctgcaccgccttctggagaagcggcaggaaggcgcggagacggtggagctgagtgcggatgggcgcccggtgaccacgcagacccgggacccgccggtggtggactgcacctgcttcggcctccctcgccgctacattatcgccatcatgagtggtctgggcttctgcatcagctttggcatccgctgcaacctgggcgtggccatcgtctccatggtcaataacagcacgacccaccgcgggggccacgtggtggtgcagaaagcccagttcagctgggatccagagactgtcggcctcatacacggctcctttttctggggctacattgtcactcagattccaggaggatttatctgtcaaaaatttgcagccaacagagttttcggctttgctattgtggcaacatccactctaaacatgctgatcccctcagctgcccgcgtccactatggctgtgtcatcttcgtgaggatcctgcaggggttggtagagggggtcacataccccgcctgccatgggatctggagcaaatgggccccacccttagaacggagtcgcctggcgacgacagccttttgtggttcctatgctggggcggtggtcgcgatgcccctcgccggggtccttgtgcagtactcaggatggagctctgttttctacgtctacggcagcttcgggatcttctggtacctgttctggctgctcgtctcctacgagtcccccgcgctgcaccccagcatctcggaggaggagcgcaagtacatcgaggacgccatcggagagagcgcgaaactcatgaaccccctcacgaagtttagcactccctggcggcgcttcttcacgtctatgccagtctatgccatcatcgtggccaacttctgccgcagctggacgttctacctgctgctcatctcccagcccgactacttcgaagaagtgttcggcttcgagatcagcaaggtaggcctggtgtccgcgctgccccacctggtcatgaccatcatcgtgcccatcggcggccagatcgcggacttcctgcggagccgccgcatcatgtccaccaccaacgtgcgcaagttgatgaactgcggaggcttcggcatggaagccacgctgctgttggtggtcggctactcgcactccaagggcgtggccatctccttcctggtcctagccgtgggcttcagcggcttcgccatctctgggttcaacgtgaaccacctggacatagccccgcgctacgccagcatcctcatgggcatctccaacggcgtgggcacactgtcgggcatggtgtgccccatcatcgtgggggccatgactaagcacaagactcgggaggagtggcagtacgtgttcctaattgcctccctggtgcactatggaggtgtcatcttctacggggtctttgcttctggagagaagcagccgtgggcagagcctgaggagatgagcgaggagaagtgtggcttcgttggccatgaccagctggctggcagtgacgacagcgaaatggaggatgaggctgagcccccgggggcaccccctgcacccccgccctcctatggggccacacacagcacatttcagccccccaggcccccaccccctgtccgggactactgaccatgtgcctcccactgaatggcagtttccaggacctccattccactcatctctggcctgagtgacagtgtcaaggaaccctgctcctctctgtcctgcctcaggcctaagaagcactctcccttgttcccagtgctgtcaaatcctctttccttcccaattgcctctcaggggtagtgaagctgcagactgacagtttcaaggatacccaaattcccctaaaggttccctctccacccgttctgcctcagtggtttcaaatctctcctttcagggctttatttgaatggacagttcgacctcttactctctcttgtggttttgaggcacccacaccccccgctttcctttatctccagggactctcaggctaacctttgagatcactcagctcccatctcctttcagaaaaattcaaggtcctcctctagaagtttcaaatctctcccaactctgttctgcatcttccagattggtttaaccaattactcgtccccgccattccagggattgattctcaccagcgtttctgatggaaaatggcgggaattcctgcagcccgggggatccact

SEQ ID NO: 7 - DNA, 20 bp; single-stranded, linear
  primer for hPiT-1 1..20
  hypothetical: NO
  anti-sense: NO
cagttcagtcaagccgtcag

SEQ ID NO: 8 - DNA, 20 bp; single-stranded, linear
  complementary strand primer for hPiT-1 1..20
  hypothetical: NO
  anti-sense: NO
ccagccaacagacacaacag

SEQ ID NO: 9 - DNA, 20 bp; single-stranded, linear
  primer for hPiT-1 1..20
  hypothetical: NO
  anti-sense: NO
acaacgagacggtggagact

SEQ ID NO: 10 - DNA, 20 bp; single-stranded, linear
  complementary strand primer for hPiT-2 1..20
  hypothetical: NO
  anti-sense: NO
tgcggtgtagcaggtgtaac

SEQ ID NO: 11 - DNA, 20 bp; single-stranded, linear
  primer for hBNPI 1..20
  hypothetical: NO
  anti-sense: NO
cctcgccgctacattatcgc

SEQ ID NO: 12 - DNA, 20 bp; single-stranded, linear
  complementary strand primer for hBNPI 1..20
  hypothetical: NO
  anti-sense: NO
cgaagcctccgcagttcatc